(19) United States
(12) Patent Application Publication
(10) Pub. No.: US 2015/0031068 A1
(43) Pub. Date: Jan. 29, 2015

(54) METHODS AND COMPOSITIONS FOR DETECTING IMMUNE SYSTEM ACTIVATION

(71) Applicants: Gary D. Glick, Ann Arbor, MI (US); Anthony W. Opipari, JR., Dexter, MI (US); Laszlo G. Boros, Los Angeles, CA (US)

(72) Inventors: Gary D. Glick, Ann Arbor, MI (US); Anthony W. Opipari, JR., Dexter, MI (US); Laszlo G. Boros, Los Angeles, CA (US)

(73) Assignee: Lycera Corporation, Ann Arbor, MI (US)

(21) Appl. No.: 14/384,036

(22) PCT Filed: Mar. 15, 2013
(86) PCT No.: PCT/US2013/031879

§ 371 Date: Sep. 9, 2014

Related U.S. Application Data
(60) Provisional application No. 61/612,584, filed on Mar. 19, 2012.

(51) Int. Cl. G01N 33/50

(52) U.S. Cl. CPC G01N 33/5091
USPC 435/29

(57) ABSTRACT

The present invention relates to methods and compositions for detecting immune system activation and/or the presence of an immune disorder in a patient. More particularly, methods and compositions for detecting immune system activation and/or the presence of an immune disorder in a patient by detecting, for example, the concentration of certain metabolites of ¹³C-labeled glutamine, ¹³C-labeled palmitate, and/or ¹³C-labeled glucose.

### CROSS REFERENCE TO RELATED APPLICATIONS

This application claims the benefit of and priority to U.S. Provisional Patent Application Ser. No. 61/612,584, filed Mar. 19, 2012, the contents of which are hereby incorporated by reference.

### FIELD OF THE INVENTION

The invention provides methods and compositions for detecting immune system activation and medical conditions featuring immune system activation. Detection methods are provided that comprise determining, for example, the amount of certain metabolites of ¹³C-labeled glutamine, ¹³C-labeled palmitate, and/or ¹³C-labeled glucose in a sample in order to detect the presence of immune system activation and/or certain medical conditions in a subject.

### BACKGROUND

Detection of medical conditions in a subject is an important component of providing proper medical care. Many medical conditions are associated with immune system activation. Exemplary medical conditions associated with immune system activation include, for example, graft-versus-host disease, systemic lupus erythematosus, psoriasis, Crohn's disease, and inflammatory bowel disease. In graft-versus-host disease, T cells from the graft become activated and attack the host. Systemic lupus erythematosus is an immune disorder in which the subject's immune system becomes activated and attacks the subject's own tissue, resulting in inflammation and tissue damage. In Crohn's disease, the subject's immune system becomes activated and attacks the gastrointestinal tract.

Immune system activity can be detected using various procedures reported in the literature. One procedure for detecting immune system activity involves obtaining a blood sample from a subject, treating the blood sample with an antibody that binds to an antigen produced during periods of immune system activation, and measuring for the presence of a complex formed by the antibody and antigen produced during periods of immune system activation. However, there are limitations to using such antibody-antigen binding assays as part of a low-cost, robust procedure for detecting the presence of immune system activation and medical conditions associated therewith in a subject.

The need exists for new methods and compositions for detecting the presence of immune system activation and medical conditions associated therewith in a subject. In particular, the need exists for diagnostic methods that are more suitable for widespread commercial use by physicians and other medical professionals in clinical settings. The present invention satisfies this need and provides other related advantages.

### SUMMARY

The invention provides methods and compositions for detecting immune system activation and/or the presence of medical conditions featuring immune system activation in a subject. The invention is based, in part, on the discovery that the metabolism of glutamine, palmitate, and glucose in subjects having an activated immune system is different than metabolism of the aforementioned compounds in healthy subjects. Features of the metabolite profile described herein for subjects having received one or more of ¹³C-labeled glutamine, ¹³C-labeled palmitate, and ¹³C-labeled glucose are used to detect and diagnose immune system activation and/or the presence of a medical condition (e.g., an immune disorder) featuring immune system activation in a subject. In addition, methods are provided for determining whether certain medical disorders are in an active state or dormant state, and methods are provided for identifying agents that have efficacy in treating medical conditions featuring immune system activation. Further aspects and embodiments of the invention are described below.

One aspect of the invention provides a method of determining immune system activation in a subject. The method comprises one or more of the following steps:
(a) measuring the amount of ¹³CO₂ in a test sample obtained from a subject that received ¹³C-labeled glutamine; and comparing (i) the amount of ¹³CO₂ in the test sample to (ii) a control, wherein a greater amount of ¹³CO₂ in the test sample than the control indicates immune system activation in the subject;
(b) measuring the amount of ¹³C-labeled ribose in a population of cells selected from the group consisting of lymphocytes and immune cells, wherein said population of cells is obtained from a subject that received ¹³C-labeled glutamine; and comparing (i) the amount of ¹³C-labeled ribose in said population of cells to (ii) a control, wherein a greater amount of ¹³C-labeled ribose in said population of cells than the control indicates immune system activation in the subject;
(c) measuring the amount of ¹³C-labeled glutamine in a test sample obtained from a subject that received ¹³C-labeled glutamine; and comparing (i) the amount of ¹³C-labeled glutamine in the test sample to (ii) a control, wherein a greater amount of ¹³C-labeled glutamine in the test sample than the control indicates immune system activation in the subject; and
(d) measuring the amount of total glutamine in a test sample obtained from a subject that received glutamine; and comparing (i) the amount of total glutamine in the test sample to (ii) a control, wherein less total glutamine in the test sample than the control indicates immune system activation in the subject.

Another aspect of the invention provides a method of determining immune system activation in a subject, where the method comprises one or more of the following steps:
(a) measuring the amount of ¹³CO₂ in a test sample obtained from a subject that received ¹³C-labeled palmitate; and comparing (i) the amount of ¹³CO₂ in the test sample to (ii) a control, wherein a greater amount of ¹³CO₂ in the test sample than the control indicates immune system activation in the subject; and
(b) measuring the molar percent enrichment in [4,5-¹³C₂]-L-glutamate in a test sample obtained from a subject that received ¹³C-labeled palmitate; and comparing (i) the molar percent enrichment in [4,5-¹³C₂]-L-glutamate in the test sample to (ii) a control, wherein greater molar percent enrichment in [4,5-¹³C₂]-L-glutamate in the test sample compared to the control indicates immune system activation in the subject.

In certain embodiments, the methods described above further comprise the steps of: (a-1) measuring the amount of ¹³CO₂ in a test sample obtained from a subject that received ¹³C-labeled glucose; and (b-1) comparing (i) the amount of ¹³CO₂ in the test sample to (ii) a control, wherein a greater amount of ¹³CO₂ in the test sample than the control indicates immune system activation in the subject.

The test sample obtained from the subject is a sample of biological material containing a metabolite (e.g., ¹³CO₂) of the ¹³C-labeled compound (e.g., ¹³C-labeled glutamine, ¹³C-labeled palmitate, and ¹³C-labeled glucose) administered to the subject. Exemplary test samples include, for example, (i) blood plasma comprising ¹³CO₂, and (ii) a population of T-cells comprising ¹³CO₂. Additional exemplary test samples include a population of lymphocytes and a population of immune cells, each of which are obtained from a subject that has received a ¹³C-labeled compound (e.g., ¹³C-labeled glutamine, ¹³C-labeled palmitate, and ¹³C-labeled glucose). In certain embodiments, the population of T-cells obtained from a subject is a population of T-cells obtained from blood (e.g., blood in the subject's circulatory system), bone marrow, cells from bronchioalveolar lavage, and cells from a biopsy of a lymph node, joint space, skin, intestine, and other tissue that contains T cells. In certain other embodiments, the population of T-cells is obtained from the spleen of the subject. In certain embodiments, test sample is blood plasma, such as blood plasma obtained from an artery or a vein.

Another aspect of the invention provides a method of determining whether a subject has a medical condition featuring immune system activation. An exemplary medical condition featuring immune system activation is an immune disorder, such as chronic graft-versus-host disease, acute graft-versus-host disease, rheumatoid arthritis, psoriasis, Crohn's disease, inflammatory bowel disease, multiple sclerosis, systemic lupus erythematosus, and Celiac Sprue. The method comprises one or more of the following steps:
(a) measuring the amount of ¹³CO₂ in a test sample obtained from a subject that received ¹³C-labeled glutamine; and comparing (i) the amount of ¹³CO₂ in the test sample to (ii) a control, wherein a greater amount of ¹³CO₂ in the test sample than the control indicates that the subject has the medical condition;
(b) measuring the amount of ¹³C-labeled ribose in a population of cells selected from the group consisting of lymphocytes and immune cells, wherein said population of cells is obtained from a subject that received ¹³C-labeled glutamine; and comparing (i) the amount of ¹³C-labeled ribose in said population of cells to (ii) a control, wherein a greater amount of ¹³C-labeled ribose in said population of cells than the control indicates that the subject has the medical condition;
(c) measuring the amount of ¹³C-labeled glutamine in a test sample obtained from a subject that received ¹³C-labeled glutamine; and comparing (i) the amount of ¹³C-labeled glutamine in the test sample to (ii) a control, wherein a greater amount of ¹³C-labeled glutamine in the test sample than the control indicates that the subject has the medical condition; and
(d) measuring the amount of total glutamine in a test sample obtained from a subject that received glutamine; and comparing (i) the amount of total glutamine in the test sample to (ii) a control, wherein less total glutamine in the test sample than the control indicates that the subject has the medical condition.

Another aspect of the invention provides a method of determining whether a subject has a medical condition featuring immune system activation, based on the metabolic profile of the subject after receiving ¹³C-labeled palmitate. The method comprises one or more of the following steps:
(a) measuring the amount of ¹³CO₂ in a test sample obtained from a subject that received ¹³C-labeled palmitate; and comparing (i) the amount of ¹³CO₂ in the test sample to (ii) a control, wherein a greater amount of ¹³CO₂ in the test sample than the control indicates that the subject has the medical condition; and
(b) measuring the molar percent enrichment in [4,5-¹³C₂]-L-glutamate in a test sample obtained from a subject that received ¹³C-labeled palmitate; and comparing (i) the molar percent enrichment in [4,5-¹³C₂]-L-glutamate in the test sample to (ii) a control, wherein greater molar percent enrichment in [4,5-¹³C₂]-L-glutamate in the test sample compared to the control indicates that the subject has the medical condition.

In certain embodiments, the methods described above further comprise the steps of: (a-1) measuring the amount of ¹³CO₂ in a test sample obtained from a subject that received ¹³C-labeled glucose; and (b-1) comparing (i) the amount of ¹³CO₂ in the test sample to (ii) a control, wherein a greater amount of ¹³CO₂ in the test sample than the control indicates that the subject has the medical condition.

Another aspect of the invention provides a method of determining whether a subject has an immune disorder, based on the metabolic profile of the subject after receiving ¹³C-labeled glucose. The method comprises the steps of: (a) measuring the amount of ¹³CO₂ in a test sample obtained from a subject that received ¹³C-labeled glucose; and (b) comparing (i) the amount of ¹³CO₂ in the test sample to (ii) a control, wherein a greater amount of ¹³CO₂ in the test sample than the control indicates that the subject has said immune disorder. Particular immune disorders include chronic graft-versus-host disease, acute graft-versus-host disease, rheumatoid arthritis, psoriasis, Crohn's disease, inflammatory bowel disease, multiple sclerosis, Celiac Sprue, idiopathic thrombocytopenic thrombotic purpura, myasthenia gravis, Sjogren's syndrome, scleroderma, ulcerative colitis, asthma, uveitis, epidermal hyperplasia, cartilage inflammation, bone degradation, arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, dermatomyositis, psoriatic arthritis, vasculitis, myolitis, polymyolitis, dermatomyolitis, osteoarthritis, polyarteritis nodossa, Wegener's granulomatosis, arteritis, polymyalgia rheumatica, sarcoidosis, sclerosis, primary biliary sclerosis, sclerosing cholangitis, dermatitis, atopic dermatitis, atherosclerosis, Still's disease, chronic obstructive pulmonary disease, Guillain-Barre disease, Type I diabetes mellitus, Graves' disease, Addison's disease, Raynaud's phenomenon, lupus nephritis, glomerular nephritis, and autoimmune hepatitis. In certain embodiments, the test sample is a population of spleen cells. In certain other embodiments, the test sample is a population of T cells, such as T-cells obtained from blood (e.g., blood in the subject's circulatory system), bone marrow, cells from bronchioalveolar lavage, and cells from a biopsy of a lymph node, joint space, skin, intestine, and other tissue that contains T cells. In yet other embodiments, the test sample is a population of lymphocytes or a population of immune cells from the subject.

### BRIEF DESCRIPTION OF FIGURES

FIG. 1 depicts bar graphs showing (A) the abundance of ¹³CO₂ in blood plasma, (B) the percentage total glutamine that was uniformly ¹³C-labeled in a sample of blood plasma, and (C) the amount of total glutamine in blood plasma of mice (either allo, host, or donor mice) that received ¹³C(5)-glutamine

FIG. 2 is a bar graph showing the percentage of ¹³C-labeled ribose in B6 Donor T cells obtained from mice (either allo or naive mice) that received ¹³C(5)-glutamine.

FIG. 3 is a bar graph showing the percentage of ¹³C-labeled ribose in B6D2 Host T cells obtained from mice (either allo or naive mice) that received ¹³C(5)-glutamine.

FIG. 4 depicts bar graphs showing (A) the change in abundance of ¹³C-labeled CO₂ in B6 Donor T cells obtained from mice (either allo or naive mice) that received ¹³C-glucose, and (B) the change in abundance of ¹³C-labeled CO₂ in B6 Donor T cells obtained from mice (either allo or naive mice) that received ¹³C-palmitate.

FIG. 5 depicts bar graphs showing (A) the change in abundance of ¹³C-labeled CO₂ in B6D2 Host T cells obtained from mice (either allo or naive mice) that received ¹³C-glucose, and (B) the change in abundance of ¹³C-labeled CO₂ in B6D2 Host T cells obtained from mice (either allo or naive mice) that received ¹³C-palmitate.

FIG. 6 is a bar graph showing the molar percent enrichment in [4,5-¹³C₂]-L-glutamate in B6 Donor T cells obtained from mice (either allo or naive mice) that received ¹³C-palmitate.

FIG. 7 is a bar graph showing the molar percent enrichment in [4,5-¹³C₂]-L-glutamate in B6D2 Host T cells obtained from mice (either allo or naive mice) that received ¹³C-palmitate.

### DETAILED DESCRIPTION OF THE INVENTION

The invention provides methods and compositions for detecting immune system activation and medical conditions featuring immune system activation in a subject. The invention is based, in part, on the discovery that the metabolism of glutamine, palmitate, and glucose in subjects having an activated immune system is different than metabolism of the aforementioned compounds in healthy subjects. For example, activation of cells of the immune system results in metabolic adaptations in these cells that provide energy and biochemical substrates necessary to meet the increased demands associated with immune cell activation. Activated T cells increase oxidative metabolism for production of ATP in subjects with immune disease.

Features of the metabolite profile described herein for subjects having received one or more of ¹³C-labeled glutamine, ¹³C-labeled palmitate, and ¹³C-labeled glucose are used to detect immune system activation and to detect the presence of a medical condition (e.g., an immune disorder) featuring immune system activation in a subject. In addition, methods are provided for (i) determining whether certain medical disorders are in an active state or dormant state, (ii) identifying agents that have efficacy in treating medical conditions featuring immune system activation, (iii) determining whether a therapeutic intervention to treat an immune disorder is working effectively, (iv) determining what amount of a therapeutic agent qualifies as an effective amount to treat an immune disorder, and (v) determining an appropriate dosing regimen for treating an immune disorder using a therapeutic agent.

Various aspects of the invention are set forth below in sections; however, aspects of the invention described in one particular section are not to be limited to any particular section. Further, when a variable is not accompanied by a definition, the previous definition of the variable controls.

### DEFINITIONS

To facilitate an understanding of the present invention, a number of terms and phrases are defined below.

The terms “a,” “an” and “the” as used herein mean “one or more” and include the plural unless the context is inappropriate.

The term “glutamine” refers to the following compound and includes salts thereof and, unless specified otherwise, includes where one or more of the atoms may be artificially enriched in a particular isotope (such as ¹³C) having the same atomic number:

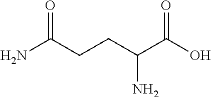

In certain embodiments, the glutamine is in the form of a free base. In certain other embodiments, the glutamine is L-glutamine. In yet other embodiments, the glutamine is L-glutamine in the form of a free base. For clarity, the term “total glutamine” refers to sum of (i) the amount of isotopically enriched glutamine (e.g., ¹³C-labeled glutamine) or a salt thereof and (ii) the amount of glutamine that is not isotopically enriched or a salt thereof. Glutamate refers to the following compound where M⁺ is a cation (e.g., an alkali metal cation such as Na⁺ or K⁺, or an organic positively charged species such as NH₄⁺):

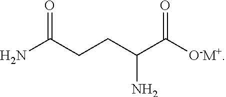

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal hydroxides, alkaline earth metals hydroxides, ammonia, and compounds of formula NW₄⁺, wherein W is C₁₋₄ alkyl, and the like. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

It will be noted that the structure of some of the compounds of the invention includes asymmetric carbon atoms. It is to be understood that the isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Furthermore, the structures and other compounds and moieties discussed in this application also include all tautomers thereof. Alkenes can include either the E- or Z-geometry, where appropriate.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Methods for Determining Immune System Activation Using ¹³C-Labeled Glutamine

One aspect of the invention provides methods and compositions for detecting immune system activation in a subject that has received ¹³C-labeled glutamine. The method involves obtaining a test sample (e.g., a blood sample) from a subject who has received ¹³C-labeled glutamine, and analyzing the test sample to determine if it contains more ¹³CO₂ than a control. A greater amount of ¹³CO₂ in the test sample relative to the control is indicative of immune system activation. In a separate test, a population of T-cells can be harvested from a subject who has received ¹³C-labeled glutamine, and the population of T-cells can be analyzed to determine the amount of ¹³C-labeled ribose in the population of T-cells. A greater amount of ¹³C-labeled ribose in the population of T-cells harvested from the subject than the amount of ¹³C-labeled ribose in the population of T-cells from a control indicates immune system activation. Lymphocytes and immune cells can be used in lieu of T-cells. The control is a benchmark value established by subjecting healthy subject(s) to approximately the same conditions as the subject from which the test sample was obtained. Differences in the metabolism of glutamine in subjects with immune system activation compared to subjects lacking immune system activation (i.e., healthy subjects) give rise to differences in the metabolic profile observed for the two types of subjects, and these differences can be used to identify immune system activation in a subject. Additional features of the metabolic profile in subjects with immune system activation who have received ¹³C-labeled glutamine are described below.

Another feature indicative of immune system activation in a subject is an increased amount of ¹³C-labeled glutamine in the test sample (e.g., a blood sample) obtained from a subject that has received ¹³C-labeled glutamine compared to a control (e.g., a blood sample taken from a healthy subject having received ¹³C-labeled glutamine).

Still another feature indicative of immune system activation in a subject is a reduction in the amount of total glutamine in a test sample (e.g., a blood sample) taken from a subject that has received glutamine compared to a control (e.g., a blood sample taken a healthy subject that has received glutamine).

The above indicia of immune system activation may be used individually or collectively to detect immune system activation in a subject. Further, the above indicia of immune system activation for a subject that has received ¹³C-labeled glutamine may be used in combination with indicia of immune system activation for a subject that has also received ¹³C-labeled palmitate and/or ¹³C-labeled glucose, as described in Sections II and III below.

Accordingly, one aspect of the invention provides a method of determining immune system activation in a subject. The method comprises one or more of the following steps:
(a) measuring the amount of ¹³CO₂ in a test sample obtained from a subject that received ¹³C-labeled glutamine; and comparing (i) the amount of ¹³CO₂ in the test sample to (ii) a control, wherein a greater amount of ¹³CO₂ in the test sample than the control indicates immune system activation in the subject;
(b) measuring the amount of ¹³C-labeled ribose in a population of cells selected from the group consisting of lymphocytes and immune cells, wherein said population of cells is obtained from a subject that received ¹³C-labeled glutamine; and comparing (i) the amount of ¹³C-labeled ribose in said population of cells to (ii) a control, wherein a greater amount of ¹³C-labeled ribose in said population of cells than the control indicates immune system activation in the subject;
(c) measuring the amount of ¹³C-labeled glutamine in a test sample obtained from a subject that received ¹³C-labeled glutamine; and comparing (i) the amount of ¹³C-labeled glutamine in the test sample to (ii) a control, wherein a greater amount of ¹³C-labeled glutamine in the test sample than the control indicates immune system activation in the subject; and
(d) measuring the amount of total glutamine in a test sample obtained from a subject that received glutamine; and comparing (i) the amount of total glutamine in the test sample to (ii) a control, wherein less total glutamine in the test sample than the control indicates immune system activation in the subject.

In a more specific embodiment, the invention provides a method of determining immune system activation in a subject, where the method comprises one or more of the following steps:
(a) measuring the amount of ¹³CO₂ in a test sample obtained from a subject that received ¹³C-labeled glutamine; and comparing (i) the amount of ¹³CO₂ in the test sample to (ii) a control, wherein a greater amount of ¹³CO₂ in the test sample than the control indicates immune system activation in the subject;
(b) measuring the amount of ¹³C-labeled ribose in a population of T-cells obtained from a subject that received ¹³C-labeled glutamine; and comparing (i) the amount of ¹³C-labeled ribose in a population of T-cells to (ii) a control, wherein a greater amount of ¹³C-labeled ribose in a population of T-cells than the control indicates immune system activation in the subject;
(c) measuring the amount of ¹³C-labeled glutamine in a test sample obtained from a subject that received ¹³C-labeled glutamine; and comparing (i) the amount of ¹³C-labeled glutamine in the test sample to (ii) a control, wherein a greater amount of ¹³C-labeled glutamine in the test sample than the control indicates immune system activation in the subject; and
(d) measuring the amount of total glutamine in a test sample obtained from a subject that received glutamine; and comparing (i) the amount of total glutamine in the test sample to (ii) a control, wherein less total glutamine in the test sample than the control indicates immune system activation in the subject.

In certain embodiments, the method comprises step (a). In certain other embodiments, the method comprises step (b). In certain other embodiments, the method comprises step (c). In certain other embodiments, the method comprises step (d). In certain other embodiments, the method comprises steps (a) and (b). In certain other embodiments, the method comprises steps (a) and (c). In certain other embodiments, the method comprises steps (a) and (d).

### Test Sample

The test sample obtained from the subject is a sample of biological material containing a metabolite (e.g., ¹³CO₂) of the ¹³C-labeled glutamine administered to the subject. Exemplary test samples include, for example, (i) blood plasma comprising ¹³CO₂, and (ii) a population of T-cells comprising ¹³CO₂. Additional exemplary test samples include a population of lymphocytes and a population of immune cells. In certain embodiments, the population of T-cells obtained from a subject is a population of T-cells obtained from blood (e.g., blood in the subject's circulatory system), bone marrow, cells from bronchioalveolar lavage, or cells from biopsy of a lymph node, joint space, skin, intestine, and other tissue that contains T cells. In certain other embodiments, the population of T-cells is obtained from the spleen of the subject. In certain embodiments, test sample is blood plasma, such as blood plasma obtained from an artery (such as a cardiac artery) or a vein.

In certain embodiments, the population of cells used in a step measuring the amount of ¹³C-labeled ribose is a population of T-cells. Such population of T-cells may be, for example, a population of T-cells obtained from blood, bone marrow, cells from bronchioalveolar lavage, or cells from a biopsy of a lymph node, a joint space, skin, or intestine of the subject.

### ¹³C-Labeled Glutamine

The ¹³C-labeled glutamine contains an amount of ¹³C sufficient to permit detection of ¹³C-labeled metabolites (e.g., ¹³CO₂) in a test sample obtained from a subject. In certain embodiments, the ¹³C-labeled glutamine is glutamine where each carbon position contains ¹³C in an abundance of at least 75% wt/wt. In certain other embodiments, the ¹³C-labeled glutamine is glutamine where each carbon position contains ¹³C in an abundance of at least 98% wt/wt. In certain other embodiments, the ¹³C-labeled glutamine is glutamine where at least one-half of the carbon positions contain ¹³C in an abundance of at least 50% wt/wt, 75% wt/wt, 90% wt/wt, 95% wt/wt, or 99% wt/wt. In certain other embodiments, the ¹³C-labeled glutamine is glutamine where at least 75% of the carbon positions contain ¹³C in an abundance of at least 50% wt/wt, 75% wt/wt, 90% wt/wt, 95% wt/wt, or 99% wt/wt.

In certain embodiments, the ¹³C-labeled glutamine is in the form of a free base. In certain embodiments, the ¹³C-labeled glutamine is in the form of a hydrochloric acid salt.

### Collection of Test Sample

The test sample is obtained from the subject after a time period sufficient for the subject to metabolize at least some of the ¹³C-labeled glutamine administered. In certain embodiments, the test sample is obtained from the subject during a time period of about 5 minutes to about 3 hours after ¹³C-labeled glutamine was administered to the subject. In certain other embodiments, the test sample is obtained from the subject during a time period of about 30 minutes to about 90 minutes after ¹³C-labeled glutamine was administered to the subject. In certain other embodiments, the test sample is obtained from the subject at about 60 minutes after ¹³C-labeled glutamine was administered to the subject.

### Administration & Dosage of ¹³C-Labeled Glutamine

The ¹³C-labeled glutamine is administered to the subject by means sufficient to deliver the ¹³C-labeled glutamine so that at least some of the ¹³C-labeled glutamine can be metabolized by the subject. In certain embodiments, the subject receives ¹³C-labeled glutamine by oral administration or injection, such as intravenous injection, intramuscular injection, intraarterial injection, intracapsular injection, subcutaneous injection, or subcapsular injection. In certain other embodiments, the subject receives ¹³C-labeled glutamine by intraperitoneal injection.

The ¹³C-labeled glutamine is generally administered in an amount sufficient to provide metabolites of ¹³C-labeled glutamine in an amount sufficient for detection by usual analytical methods. For example, in certain embodiments, the subject receives ¹³C-labeled glutamine at a dosage of from about 0.1 g/kg to about 10 g/kg, from about 0.1 g/kg to about 5 g/kg, from about 0.5 g/kg to about 2 g/kg, or from about 0.5 g/kg to about 1.5 g/kg.

### T-Cell Populations

A T-cell population may be obtained from the patient by biopsy of tissue containing T-cells. In certain embodiments, the population of T-cells obtained from a subject is a population of T-cells obtained from blood (e.g., blood in the subject's circulatory system), bone marrow, cells from bronchioalveolar lavage, and cells from a biopsy of a lymph node, joint space, skin, intestine, and other tissue that contains T cells. In certain other embodiments, the population of T-cells obtained from a subject is a population of T-cells obtained from the spleen of the subject. The quantity of T-cells obtained from the subject should be sufficient to permit detection of metabolites of ¹³C-labeled glutamine by usual analytical methods. In certain embodiments, the population of T-cells contains at least 100 T-cells, 1,000 T-cells, or 10,000 T-cells.

### The Control

The control is a benchmark value established by subjecting healthy subject(s) to approximately the same conditions as the subject from which the test sample was obtained. In particular, the control is the average amount of corresponding analyte in samples taken from a population of healthy subjects subjected to approximately the same conditions as the subject from which the test sample was taken. For instance, when evaluating the amount of ¹³CO₂ in a test sample from a subject that received ¹³C-labeled glutamine in order to determine immune system activation, the control is the average amount of ¹³CO₂ (i.e., the corresponding analyte) in samples taken from a population of healthy subjects subjected to approximately the same conditions as the subject from which the test sample was taken. The test sample is desirably the same type of biological material (e.g., blood plasma) as the samples taken from the population of healthy subjects used to establish the control. When evaluating the amount of ¹³C-labeled ribose in a population of T-cells obtained from a subject that received ¹³C-labeled glutamine in order to determine immune system activation, the control is the average amount of ¹³C-labeled ribose (i.e., the corresponding analyte) in a population of T-cells obtained from a population of healthy subjects subjected to approximately the same conditions as the subject from which the test sample was taken. When evaluating the amount of ¹³C-labeled glutamine in a test sample from a subject that received ¹³C-labeled glutamine in order to determine immune system activation, the control is the average amount of ¹³C-labeled glutamine (i.e., the corresponding analyte) in samples taken from a population of healthy subjects subjected to approximately the same conditions as the subject from which the test sample was taken. Similarly, when evaluating the total amount of glutamine in a test sample from a subject that received glutamine in order to determine immune system activation, the control is the average amount of total glutamine (i.e., the corresponding analyte) in samples taken from a population of healthy subjects subjected to approximately the same conditions as the subject from which the test sample was taken.

### Comparing the Amount of Analyte in Test Sample to the Control

The amount of analyte (e.g., ¹³CO₂) in the test sample can be compared to the control using statistical procedures known in the art. For example, in certain embodiments, the comparing is performed by statistical analysis using the Student's T-test.

### Determining the Amount of Analyte in Test Sample

The amount of analyte in biological samples (e.g., the test sample obtained from a subject) can be determined using analytical techniques known in the art. For example, in certain embodiments, measuring the amount of ¹³CO₂ in a test sample comprises analysis of the test sample using mass spectrometry to identify ¹³CO₂.

### Immune System Activation

Immune system activation can be also determined by detecting for the presence of multiple, different types of biological markers. For example, in certain embodiments, the immune system activation comprises T-cell activation. In certain other embodiments, the immune system activation comprises the activation of one or more of T-cells, B-cells, monocytes, macrophages, peripheral blood mononuclear cells, or peripheral blood mononuclear leukocytes. In yet other embodiments, the immune system activation comprises the activation of dendritic cells.

### Further Methods for Determining Immune System Activation

The immune system activation detection methods described above can be combined with immune system activation detection steps that comprise detection of a metabolite of ¹³C-labeled palmitate and/or a metabolite of ¹³C-labeled glucose. Procedures for detecting immune system activation based on metabolites of ¹³C-labeled palmitate are described in Section II below. The steps for detecting immune system activation based on metabolites of ¹³C-labeled palmitate can be added to those used to determine immune system activation based on analysis of metabolites of ¹³C-labeled glutamine.

Additionally, when it is desirable to supplement the above detection methods to include analysis of the metabolites of ¹³C-labeled glucose, the method further comprises the steps of: (a-1) measuring the amount of ¹³CO₂ in a test sample obtained from a subject that received ¹³C-labeled glucose; and (b-1) comparing (i) the amount of ¹³CO₂ in the test sample to (ii) a control, wherein a greater amount of ¹³CO₂ in the test sample than the control indicates immune system activation in the subject. In certain embodiments, the test sample is a population of T cells, such as a population of T cells obtained from the spleen of the subject, or population of T-cells obtained from blood (e.g., blood in the subject's circulatory system), bone marrow, cells from bronchioalveolar lavage, and cells from biopsies of lymph nodes, joint spaces, skin, intestine, and other tissues that contain T cells.

In certain other embodiments, the method may further comprise one or more of the following: (i) selecting a subject having at least one indicia of an immune disorder, (ii) administering to a subject one or more of ¹³C-labeled glutamine, ¹³C-labeled palmitate, and ¹³C-labeled glucose, (iii) obtaining a test sample and/or population of cells from a subject who has received one or more of ¹³C-labeled glutamine, ¹³C-labeled palmitate, and ¹³C-labeled glucose, (iv) preserving said test sample and/or population of cells until said test sample and/or population of cells can be measured for the presence of metabolites, and (v) administering a therapeutic agent for treating an immune disorder to those subjects that tested positive for immune system activation (where optionally the therapeutic agent is selected based on the magnitude of immune system activation of the subject, as determined by, for example, the magnitude of the difference in abundance of a tested metabolite in a test sample (or population of cells) compared to the control).

### II. Methods for Determining Immune System Activation Using ¹³C-Labeled Palmitate

One aspect of the invention provides methods and compositions for detecting immune system activation in a subject that has received ¹³C-labeled palmitate. The method involves obtaining a test sample (e.g., a population of T cells) from the subject who has received ¹³C-labeled palmitate, and analyzing the test sample to determine if it contains more ¹³CO₂ than a control. A greater amount of ¹³CO₂ in the test sample relative to the control is indicative of immune system activation. The above indicia of immune system activation obtained for a subject that has received ¹³C-labeled palmitate may be used in combination with indicia of immune system activation for a subject that has received ¹³C-labeled glutamine and/or ¹³C-labeled glucose, as described in Section I above and Section III below, respectively. Alternatively or in addition, the method can comprise measuring the molar percent enrichment in [4,5-¹³C₂]-L-glutamate in a test sample obtained from a subject that received ¹³C-labeled palmitate, and comparing (i) the molar percent enrichment in [4,5-¹³C₂]-L-glutamate in the test sample to (ii) a control. A greater molar percent enrichment in [4,5-¹³C₂]-L-glutamate in the test sample compared to the control indicates immune system activation. The molar percent enrichment of [4,5-¹³C₂]-L-glutamate is defined as the amount of [4,5-¹³C₂]-L-glutamate in a sample divided by the total amount of all ¹³C-labeled glutamate isotopomers in the sample.

Accordingly, one aspect of the invention provides a method of determining immune system activation in a subject. The method comprising one or more of the following steps:
(a) measuring the amount of ¹³CO₂ in a test sample obtained from a subject that received ¹³C-labeled palmitate; and comparing (i) the amount of ¹³CO₂ in the test sample to (ii) a control, wherein a greater amount of ¹³CO₂ in the test sample than the control indicates immune system activation in the subject; and
(b) measuring the molar percent enrichment in [4,5-¹³C₂]-L-glutamate in a test sample obtained from a subject that received ¹³C-labeled palmitate; and comparing (i) the molar percent enrichment in [4,5-¹³C₂]-L-glutamate in the test sample to (ii) a control, wherein greater molar percent enrichment in [4,5-¹³C₂]-L-glutamate in the test sample compared to the control indicates immune system activation in the subject.

In certain embodiments, the method comprises step (a). In certain other embodiments, the method comprises step (b).

In a more specific embodiment, the invention provides a method of determining immune system activation in a subject, where the method comprises the steps of: (a) measuring the amount of ¹³CO₂ in a test sample obtained from a subject that received ¹³C-labeled palmitate; and (b) comparing (i) the amount of ¹³CO₂ in the test sample to (ii) a control, wherein a greater amount of ¹³CO₂ in the test sample than the control indicates immune system activation in the subject.

### Test Sample

The test sample obtained from the subject is a sample of biological material containing a metabolite (e.g., ¹³CO₂) of the ¹³C-labeled palmitate administered to the subject. Exemplary test samples include, for example, a population of T-cells comprising ¹³CO₂. Additional exemplary test samples include a population of lymphocytes and a population of immune cells. Such test samples are obtained from a subject that has received ¹³C-labeled palmitate. In certain embodiments, the population of T-cells obtained from a subject is a population of T-cells obtained from blood (e.g., blood in the subject's circulatory system), bone marrow, cells from bronchioalveolar lavage, and cells from a biopsy of a lymph node, joint space, skin, intestine, and other tissue that contains T cells. In certain other embodiments, the population of T-cells is obtained from the spleen of the subject. The test sample may also be a population of lymphocytes or immune cells obtained from a subject that received ¹³C-labeled palmitate. The population of lymphocytes or immune cells should comprise ¹³CO₂.

A population of T-cells may be obtained from the subject by biopsy of tissue (e.g., the spleen) containing T-cells. In certain embodiments, the population of T-cells obtained from a subject is a population of T-cells obtained from blood (e.g., blood in the subject's circulatory system), bone marrow, cells from bronchioalveolar lavage, and cells from a biopsy of a lymph node, joint space, skin, intestine, and other tissue that contains T cells. In certain other embodiments, the population of T-cells is obtained from the spleen of the subject. The quantity of T-cells obtained from the subject should be sufficient to permit detection of metabolites of ¹³C-labeled palmitate by usual analytical methods. In certain embodiments, the population of T-cells contains at least 100 T-cells, 1,000 T-cells, or 10,000 T-cells.

### ¹³C-Labeled Palmitate

The ¹³C-labeled palmitate contains an amount of ¹³C sufficient to permit detection of ¹³C-labeled metabolites (e.g., ¹³CO₂) in a test sample obtained from a subject. In certain embodiments, the ¹³C-labeled palmitate is palmitate where each carbon position contains ¹³C in an abundance of at least 75% wt/wt. In certain other embodiments, the ¹³C-labeled palmitate is palmitate where each carbon position contains ¹³C in an abundance of at least 98% wt/wt. In certain other embodiments, the ¹³C-labeled palmitate is palmitate where at least one-half of the carbon positions contain ¹³C in an abundance of at least 50% wt/wt, 75% wt/wt, 90% wt/wt, 95% wt/wt, or 99% wt/wt. In certain other embodiments, the ¹³C-labeled palmitate is palmitate where at least 75% of the carbon positions contain ¹³C in an abundance of at least 50% wt/wt, 75% wt/wt, 90% wt/wt, 95% wt/wt, or 99% wt/wt.

In certain other embodiments, the ¹³C-labeled palmitate is an alkali metal ¹³C-labeled palmitate salt. In certain other embodiments, the ¹³C-labeled palmitate is potassium ¹³C-labeled palmitate.

### Collection of Test Sample

The test sample is obtained from the subject after a time period sufficient for the subject to metabolize at least some of the ¹³C-labeled palmitate administered. In certain embodiments, the test sample is obtained from the subject during a time period of from about 1 hours to about 5 hours after ¹³C-labeled palmitate was administered to the subject. In certain embodiments, the test sample is obtained from the subject during a time period of from about 2 hours to about 4 hours after ¹³C-labeled palmitate was administered to the subject. In certain embodiments, the test sample is obtained from the subject about 3 hours after ¹³C-labeled palmitate was administered to the subject.

### Administration & Dosage of ¹³C-Labeled Palmitate

The ¹³C-labeled palmitate is administered to the subject by means sufficient to deliver the ¹³C-labeled palmitate so that at least some of the ¹³C-labeled palmitate can be metabolized by the subject. In certain embodiments, the subject receives ¹³C-labeled palmitate by oral administration or injection, such as intravenous injection, intramuscular injection, intraarterial injection, intracapsular injection, subcutaneous injection, or subcapsular injection. In certain embodiments, the subject receives ¹³C-labeled palmitate by oral administration.

The ¹³C-labeled palmitate is generally administered in an amount sufficient to provide metabolites of ¹³C-labeled palmitate in an amount sufficient for detection by usual analytical methods. For example, in certain embodiments, the subject receives ¹³C-labeled palmitate at a dosage of from about 0.1 g/kg to about 10 g/kg, from about 0.1 g/kg to about 5 g/kg, from about 0.5 g/kg to about 2 g/kg, or from about 0.5 g/kg to about 1.5 g/kg.

### The Control

The control is a benchmark value established by subjecting healthy subject(s) to approximately the same conditions as the subject from which the test sample was obtained. In particular, the control is the average amount of corresponding analyte in samples taken from a population of healthy subjects subjected to approximately the same conditions as the subject from which the test sample was taken. For instance, when evaluating the amount of ¹³CO₂ in a test sample from a subject that received ¹³C-labeled palmitate in order to determine immune system activation, the control is the average amount of ¹³CO₂ (i.e., the corresponding analyte) in samples taken from a population of healthy subjects subjected to approximately the same conditions as the subject from which the test sample was taken. The test sample is desirably the same type of biological material (e.g., a population of T-cells obtained from spleen tissue, blood (e.g., blood in the subject's circulatory system), bone marrow, cells from bronchioalveolar lavage, and cells from a biopsy of a lymph node, joint space, skin, intestine, and other tissue that contains T cells) as the samples taken from the population of healthy subjects used to establish the control.

### Comparing the Amount of Analyte in Test Sample to the Control

The amount of analyte (e.g., ¹³CO₂) in the test sample can be compared to the control using statistical procedures known in the art. For example, in certain embodiments, the comparing is performed by statistical analysis using the Student's T-test.

### Determining the Amount of Analyte in Test Sample

The amount of analyte in biological samples (e.g., the test sample obtained from a subject) can be determined using analytical techniques known in the art. For example, in certain embodiments, measuring the amount of ¹³CO₂ in a test sample comprises analysis of the test sample using mass spectrometry to identify ¹³CO₂.

### Immune System Activation

Immune system activation can also be determined by detecting for the presence of multiple, different types of biological markers. For example, in certain embodiments, the immune system activation comprises T-cell activation. In certain other embodiments, the immune system activation comprises the activation of one or more of T-cells, B-cells, monocytes, macrophages, peripheral blood mononuclear cells, or peripheral blood mononuclear leukocytes. In yet other embodiments, the immune system activation comprises the activation of dendritic cells.

### Further Methods for Determining Immune System Activation

The immune system activation detection methods described above can be combined with immune system activation detection steps that comprise detection of a metabolite of ¹³C-labeled glutamine and/or a metabolite of ¹³C-labeled glucose. Procedures for detecting immune system activation based on metabolites of ¹³C-labeled glutamine are described in Section I above. The steps for detecting immune system activation based on metabolites of ¹³C-labeled glutamine can be added to those used to determine immune system activation based on analysis of metabolites of ¹³C-labeled palmitate.

Additionally, when it is desirable to supplement the detection methods described above to include analysis of metabolites of ¹³C-labeled glucose, the method further comprises the steps of: (a-1) measuring the amount of ¹³CO₂ in a test sample obtained from a subject that received ¹³C-labeled glucose; and (b-1) comparing (i) the amount of ¹³CO₂ in the test sample to (ii) a control, wherein a greater amount of ¹³CO₂ in the test sample than the control indicates immune system activation in the subject.

1. Test Sample

The test sample obtained from the subject is a sample of biological material containing a metabolite (e.g., ¹³CO₂) of the ¹³C-labeled glutamine administered to the subject. Exemplary test samples include, for example, a population of T-cells comprising ¹³CO₂. Additional exemplary test samples include a population of lymphocytes and a population of immune cells, each comprising ¹³CO₂. In certain embodiments, the population of T-cells obtained from a subject is a population of T-cells obtained from blood (e.g., blood in the subject's circulatory system), bone marrow, cells from bronchioalveolar lavage, and cells from a biopsy of a lymph node, joint space, skin, intestine, and other tissue that contains T cells. In certain other embodiments, the population of T-cells is obtained from the spleen of the subject.

2. ¹³C-Labeled Glucose

The ¹³C-labeled glucose contains an amount of ¹³C sufficient to permit detection of ¹³C-labeled metabolites (e.g., ¹³CO₂) in a test sample obtained from a subject. In certain embodiments, the ¹³C-labeled glucose is glucose where each carbon position contains ¹³C in an abundance of at least 75% wt/wt. In certain embodiments, the ¹³C-labeled glucose is glucose where each carbon position contains ¹³C in an abundance of at least 98% wt/wt. In certain embodiments, the ¹³C-labeled glucose is glucose where at least one-half of the carbon positions contain ¹³C in an abundance of at least 50% wt/wt, 75% wt/wt, 90% wt/wt, 95% wt/wt, or 99% wt/wt. In certain embodiments, the ¹³C-labeled glucose is glucose where at least 75% of the carbon positions contain ¹³C in an abundance of at least 50% wt/wt, 75% wt/wt, 90% wt/wt, 95% wt/wt, or 99% wt/wt.

3. Collection of Test Sample

The test sample is obtained from the subject after a time period sufficient for the subject to metabolize at least some of the ¹³C-labeled glucose administered. In certain embodiments, the test sample is obtained from the subject during a time period of from about 5 minutes to about 3 hours after ¹³C-labeled glucose was administered to the subject. In certain other embodiments, the test sample is obtained from the subject during a time period of from about 30 minutes to about 90 minutes after ¹³C-labeled glucose was administered to the subject. In certain other embodiments, the test sample is obtained from the subject about 60 minutes after ¹³C-labeled glucose was administered to the subject.

4. Administration & Dosage of ¹³C-Labeled Glucose

The ¹³C-labeled glucose is administered to the subject by means sufficient to deliver the ¹³C-labeled glucose so that at least some of the ¹³C-labeled glucose can be metabolized by the subject. In certain embodiments, the subject receives ¹³C-labeled glucose by oral administration or injection, such as intravenous injection, intramuscular injection, intraarterial injection, intracapsular injection, subcutaneous injection, or subcapsular injection. In certain other embodiments, the subject receives ¹³C-labeled glutamine by intraperitoneal injection.

The ¹³C-labeled glucose is generally administered in an amount sufficient to provide metabolites of ¹³C-labeled glucose in an amount sufficient for detection by usual analytical methods. For example, in certain embodiments, the subject receives ¹³C-labeled glucose at a dosage of from about 0.1 g/kg to about 10 g/kg, from about 0.1 g/kg to about 5 g/kg, from about 0.5 g/kg to about 2 g/kg, or from about 0.5 g/kg to about 1.5 g/kg.

5. The Control

The control is a benchmark value established by subjecting healthy subject(s) to approximately the same conditions as the subject from which the test sample was obtained. In particular, the control is the average amount of corresponding analyte in samples taken from a population of healthy subjects subjected to approximately the same conditions as the subject from which the test sample was taken. For instance, when evaluating the amount of ¹³CO₂ in a test sample from a subject that received ¹³C-labeled glucose in order to determine immune system activation, the control is the average amount of ¹³CO₂ (i.e., the corresponding analyte) in samples taken from a population of healthy subjects subjected to approximately the same conditions as the subject from which the test sample was taken. The test sample is desirably the same type of biological material (e.g., a population of T-cells obtained from spleen tissue, blood (e.g., blood in the subject's circulatory system), bone marrow, cells from bronchioalveolar lavage, and cells from biopsies of lymph nodes, joint spaces, skin, intestine, or other tissue that contains T cells) as the samples taken from the population of healthy subjects used to establish the control.

6. Comparing the Amount of Analyte in Test Sample to the Control

The amount of analyte (e.g., ¹³CO₂) in the test sample can be compared to the control using statistical procedures known in the art. For example, in certain embodiments, the comparing is performed by statistical analysis using the Student's T-test

7. Determining the Amount of Analyte in Test Sample

The amount of analyte in biological samples (e.g., the test sample obtained from a subject) can be determined using analytical techniques known in the art. For example, in certain embodiments, measuring the amount of ¹³CO₂ in a test sample comprises analysis of the test sample using mass spectrometry to identify ¹³C-labeled carbon dioxide.

8. Optional Additional Steps

In certain other embodiments, the method may further comprise one or more of the following: (i) selecting a subject having at least one indicia of an immune disorder, (ii) administering to a subject one or more of ¹³C-labeled glutamine, ¹³C-labeled palmitate, and ¹³C-labeled glucose, (iii) obtaining a test sample and/or population of cells from a subject who has received one or more of ¹³C-labeled glutamine, ¹³C-labeled palmitate, and ¹³C-labeled glucose, (iv) preserving said test sample and/or population of cells until said test sample and/or population of cells can be measured for the presence of metabolites, and (v) administering a therapeutic agent for treating an immune disorder to those subjects that tested positive for immune system activation (where optionally the therapeutic agent is selected based on the magnitude of immune system activation of the subject, as determined by, for example, the magnitude of the difference in abundance of a tested metabolite in a test sample (or population of cells) compared to the control.)

### III. Methods for Detecting a Medical Condition Featuring Immune System Activation Using ¹³C-Labeled Using Glutamine

Another aspect of the invention provides a method of determining whether a subject has a medical condition featuring immune system activation. An exemplary medical condition featuring immune system activation is an immune disorder, such as, chronic graft-versus-host disease, acute graft-versus-host disease, rheumatoid arthritis, psoriasis, Crohn's disease, inflammatory bowel disease, multiple sclerosis, systemic lupus erythematosus, and Celiac Sprue. The method comprises one or more of the following steps:
(a) measuring the amount of ¹³CO₂ in a test sample obtained from a subject that received ¹³C-labeled glutamine; and comparing (i) the amount of ¹³CO₂ in the test sample to (ii) a control, wherein a greater amount of ¹³CO₂ in the test sample than the control indicates that the subject has the medical condition;
(b) measuring the amount of ¹³C-labeled ribose in a population of cells selected from the group consisting of lymphocytes and immune cells, wherein said population of cells is obtained from a subject that received ¹³C-labeled glutamine; and comparing (i) the amount of ¹³C-labeled ribose in said population of cells to (ii) a control, wherein a greater amount of ¹³C-labeled ribose in said population of cells than the control indicates that the subject has the medical condition;
(c) measuring the amount of ¹³C-labeled glutamine in a test sample obtained from a subject that received ¹³C-labeled glutamine; and comparing (i) the amount of ¹³C-labeled glutamine in the test sample to (ii) a control, wherein a greater amount of ¹³C-labeled glutamine in the test sample than the control indicates that the subject has the medical condition; and
(d) measuring the amount of total glutamine in a test sample obtained from a subject that received glutamine; and comparing (i) the amount of total glutamine in the test sample to (ii) a control, wherein less total glutamine in the test sample than the control indicates that the subject has the medical condition.

In a more specific embodiment, the method comprises one or more of the following steps:
(a) measuring the amount of ¹³CO₂ in a test sample obtained from a subject that received ¹³C-labeled glutamine; and comparing (i) the amount of ¹³CO₂ in the test sample to (ii) a control, wherein a greater amount of ¹³CO₂ in the test sample than the control indicates that the subject has the medical condition;
(b) measuring the amount of ¹³C-labeled ribose in a population of T-cells obtained from a subject that received ¹³C-labeled glutamine; and comparing (i) the amount of ¹³C-labeled ribose in a population of T-cells to (ii) a control, wherein a greater amount of ¹³C-labeled ribose in a population of T-cells than the control indicates that the subject has the medical condition;
(c) measuring the amount of ¹³C-labeled glutamine in a test sample obtained from a subject that received ¹³C-labeled glutamine; and comparing (i) the amount of ¹³C-labeled glutamine in the test sample to (ii) a control, wherein a greater amount of ¹³C-labeled glutamine in the test sample than the control indicates that the subject has the medical condition; and
(d) measuring the amount of total glutamine in a test sample obtained from a subject that received glutamine; and comparing (i) the amount of total glutamine in the test sample to (ii) a control, wherein less total glutamine in the test sample than the control indicates that the subject has the medical condition.

The method is contemplated to be applicable to detecting the presence of various types of medical conditions, such as, an immune disorder selected from the group consisting of chronic graft-versus-host disease, acute graft-versus-host disease, rheumatoid arthritis, psoriasis, Crohn's disease, inflammatory bowel disease, multiple sclerosis, systemic lupus erythematosus, Celiac Sprue, idiopathic thrombocytopenic thrombotic purpura, myasthenia gravis, Sjogren's syndrome, scleroderma, ulcerative colitis, asthma, uveitis, and epidermal hyperplasia.

In certain other embodiments, the medical condition featuring immune system activation is cartilage inflammation, bone degradation, arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, dermatomyositis, psoriatic arthritis, vasculitis, myolitis, polymyolitis, dermatomyolitis, osteoarthritis, polyarteritis nodossa, Wegener's granulomatosis, arteritis, polymyalgia rheumatica, sarcoidosis, sclerosis, primary biliary sclerosis, sclerosing cholangitis, dermatitis, atopic dermatitis, atherosclerosis, Still's disease, chronic obstructive pulmonary disease, Guillain-Barre disease, Type I diabetes mellitus, Graves' disease, Addison's disease, Raynaud's phenomenon, lupus nephritis, glomerular nephritis, or autoimmune hepatitis.

In certain other embodiments, the medical condition featuring immune system activation is muscular dystrophy, arthritis, traumatic brain injury, spinal cord injury, sepsis, rheumatic disease, cancer atherosclerosis, type 1 diabetes, type 2 diabetes, leptospiriosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, ischemia/reperfusion, stroke, cerebral aneurysm, angina pectoris, pulmonary disease, cystic fibrosis, acid-induced lung injury, pulmonary hypertension, asthma, chronic obstructive pulmonary disease, Sjogren's syndrome, hyaline membrane disease, kidney disease, glomerular disease, alcoholic liver disease, gut diseases, peritoneal endometriosis, skin diseases, nasal sinusitis, mesothelioma, anhidrotic ecodermal dysplasia-ID, behcet's disease, incontinentia pigmenti, tuberculosis, asthma, crohn's disease, colitis, ocular allergy, appendicitis, paget's disease, pancreatitis, periodonitis, endometriosis, inflammatory bowel disease, inflammatory lung disease, silica-induced diseases, sleep apnea, AIDS, HIV-1, autoimmune diseases, antiphospholipid syndrome, lupus, lupus nephritis, familial mediterranean fever, hereditary periodic fever syndrome, psychosocial stress diseases, neuropathological diseases, familial amyloidotic polyneuropathy, inflammatory neuropathy, parkinson's disease, multiple sclerosis, alzheimer's disease, amyotropic lateral sclerosis, huntington's disease, cataracts, or hearing loss.

In certain other embodiments, the medical condition featuring immune system activation is a head injury, uveitis, inflammatory pain, allergen induced asthma, non-allergen induced asthma, glomerular nephritis, ulcerative colitis, necrotizing enterocolitis, hyperimmunoglobulinemia D with recurrent fever (HIDS), TNF receptor associated periodic syndrome (TRAPS), cryopyrin-associated periodic syndromes, Muckle-Wells syndrome (urticaria deafness amyloidosis), familial cold urticaria, neonatal onset multisystem inflammatory disease (NOMID), periodic fever, aphthous stomatitis, pharyngitis and adenitis (PFAPA syndrome), Blau syndrome, pyogenic sterile arthritis, pyoderma gangrenosum, acne (PAPA), deficiency of the interleukin-1-receptor antagonist (DIRA), subarachnoid hemorrhage, polycystic kidney disease, transplant, organ transplant, tissue transplant, myelodysplastic syndrome, irritant-induced inflammation, plant irritant-induced inflammation, poison ivy/urushiol oil-induced inflammation, chemical irritant-induced inflammation, bee sting-induced inflammation, insect bite-induced inflammation, sunburn, burns, dermatitis, endotoxemia, lung injury, acute respiratory distress syndrome, alcoholic hepatitis, or kidney injury caused by parasitic infections.

In certain embodiments, the method comprises step (a). In certain other embodiments, the method comprises step (b). In certain other embodiments, the method comprises step (c). In certain other embodiments, the method comprises step (d). In certain other embodiments, the method comprises steps (a) and (b). In certain other embodiments, the method comprises steps (a) and (c). In certain other embodiments, the method comprises steps (a) and (d).

The parameters described in Section I relating to, for example, Test Sample, ¹³C-Labeled Glutamine, Collection of Test Sample, Administration & Dosage of ¹³C-labeled Glutamine, T-Cell Populations, The Control, Comparing the Amount of Analyte in Test Sample to the Control, and Determining the Amount of Analyte in Test Sample are contemplated to be applicable to the methods for determining whether a subject has a medical condition featuring immune system activation, and, accordingly, the parameters from Section I are reiterated here.

The medical condition detection methods described above can be combined with medical condition detection steps that comprise detection of a metabolite of ¹³C-labeled palmitate and/or a metabolite of ¹³C-labeled glucose. Procedures for detecting medical conditions based on metabolites of ¹³C-labeled palmitate are described in Section IV below. The steps for detecting medical conditions based on metabolites of ¹³C-labeled palmitate can be added to those used to detect the presence of a medical condition based on analysis of metabolites of ¹³C-labeled glutamine.

Additionally, when it is desirable to supplement the detection methods described above to include analysis of metabolites of ¹³C-labeled glucose, the method further comprises the steps of: (a-1) measuring the amount of ¹³CO₂ in a test sample obtained from a subject that received ¹³C-labeled glucose; and (b-1) comparing (i) the amount of ¹³CO₂ in the test sample to (ii) a control, wherein a greater amount of ¹³CO₂ in the test sample than the control indicates that the subject has the medical condition. In certain embodiments, the test sample used for detection of metabolites of ¹³C-labeled glucose is a population of T cells, such as T cells obtained from the spleen of the subject, or T cells obtained from blood, bone marrow, cells from bronchioalveolar lavage, or cells from a biopsy of a lymph node, a joint space, skin, or intestine of the subject. Further, the parameters described in Section II relating to Test Sample, ¹³C-Labeled Glucose, Collection of Test Sample, Administration & Dosage of ¹³C-labeled Glucose, The Control, and Determining the Amount of Analyte in Test Sample, each in connection with analysis of metabolites of ¹³C-labeled glucose, are contemplated to be applicable to the methods for determining whether a subject has a medical condition featuring immune system activation, and, accordingly, the aforementioned parameters from Section II are reiterated here.

### IV. Methods for Detecting a Medical Condition Featuring Immune System Activation Using ¹³C-Labeled Palmitate

Another aspect of the invention provides a method of determining whether a subject has a medical condition featuring immune system activation, based on the detection of metabolites of ¹³C-labeled palmitate. An exemplary medical condition featuring immune system activation is an immune disorder, such as, chronic graft-versus-host disease, acute graft-versus-host disease, rheumatoid arthritis, psoriasis, Crohn's disease, inflammatory bowel disease, multiple sclerosis, systemic lupus erythematosus, and Celiac Sprue. The method comprises one or more of the following steps:
(a) measuring the amount of ¹³CO₂ in a test sample obtained from a subject that received ¹³C-labeled palmitate; and comparing (i) the amount of ¹³CO₂ in the test sample to (ii) a control, wherein a greater amount of ¹³CO₂ in the test sample than the control indicates that the subject has the medical condition; and
(b) measuring the molar percent enrichment in [4,5-¹³C₂]-L-glutamate in a test sample obtained from a subject that received ¹³C-labeled palmitate; and comparing (i) the molar percent enrichment in [4,5-¹³C₂]-L-glutamate in the test sample to (ii) a control, wherein greater molar percent enrichment in [4,5-¹³C₂]-L-glutamate in the test sample compared to the control indicates that the subject has the medical condition.

In certain embodiments, the method comprises step (a). In certain other embodiments, the method comprises step (b).

In a more specific embodiment, the method comprises the steps of: (a) measuring the amount of ¹³CO₂ in a test sample obtained from a subject that received ¹³C-labeled palmitate; and (b) comparing (i) the amount of ¹³CO₂ in the test sample to (ii) a control, wherein a greater amount of ¹³CO₂ in the test sample than the control indicates that the subject has the medical disorder.

The parameters described in Section II relating to, for example, Test Sample, ¹³C-Labeled Palmitate, Collection of Test Sample, Administration & Dosage of ¹³C-labeled Palmitate, T-Cell Populations, The Control, Comparing the Amount of Analyte in Test Sample to the Control, and Determining the Amount of Analyte in Test Sample are contemplated to be applicable to the methods for determining whether a subject has a medical condition featuring immune system activation, and, accordingly, the parameters from Section II are reiterated here.

### V. Methods for Detecting a Medical Condition Featuring Immune System Activation Using ¹³C-Labeled Glucose

Another aspect of the invention provides a method of determining whether a subject has an immune disorder, based on detection of metabolites of ¹³C-labeled Glucose. The method comprises the steps of: (a) measuring the amount of ¹³CO₂ in a test sample obtained from a subject that received ¹³C-labeled glucose; and (b) comparing (i) the amount of ¹³CO₂ in the test sample to (ii) a control, wherein a greater amount of ¹³CO₂ in the test sample than the control indicates that the subject has said immune disorder.

The method is contemplated to be amenable to detecting various types of immune disorders, such as, graft-versus-host disease, acute graft-versus-host disease, rheumatoid arthritis, psoriasis, Crohn's disease, inflammatory bowel disease, multiple sclerosis, Celiac Sprue, idiopathic thrombocytopenic thrombotic purpura, myasthenia gravis, Sjogren's syndrome, scleroderma, ulcerative colitis, asthma, uveitis, epidermal hyperplasia, cartilage inflammation, bone degradation, arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome, juvenile dermatomyositis, juvenile psoriatic arthritis, juvenile scleroderma, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, dermatomyositis, psoriatic arthritis, vasculitis, myolitis, polymyolitis, dermatomyolitis, osteoarthritis, polyarteritis nodossa, Wegener's granulomatosis, arteritis, polymyalgia rheumatica, sarcoidosis, sclerosis, primary biliary sclerosis, sclerosing cholangitis, dermatitis, atopic dermatitis, atherosclerosis, Still's disease, chronic obstructive pulmonary disease, Guillain-Barre disease, Type I diabetes mellitus, Graves' disease, Addison's disease, Raynaud's phenomenon, glomerular nephritis, or autoimmune hepatitis.

The parameters described in the portion of Section II relating to, for example, Test Sample, ¹³C-Labeled Glucose, Collection of Test Sample, Administration & Dosage of ¹³C-labeled Glucose, T-Cell Populations, The Control, Comparing the Amount of Analyte in Test Sample to the Control, and Determining the Amount of Analyte in Test Sample, each in connection with the assay procedures involving detection of metabolites of ¹³C-labeled glucose, are contemplated to be applicable to the methods for determining whether a subject has a medical condition featuring immune system activation, and, accordingly, the parameters from Section II are reiterated here.

VI. Methods for Identifying a Compound with Efficacy Against Medical Conditions Featuring Immune System Activation

Another aspect of the invention provides a method for identifying a compound that has efficacy against a medical condition featuring immune system activation. The method comprises the steps of: administering a test compound to a subject having a medical condition featuring immune system activation, then testing for the presence of immune system activation in the subject according to any one of the methods described in Sections I and II above, wherein the absence of immune system activation in the subject is indicative that the test compound has efficacy in treating the medical condition. The method is contemplated to be applicable to the medical conditions listed in Section IV above, which are reiterated here. In certain embodiments, the step of testing for the presence of immune system activation is performed at least 1 hour, 5 hours, 12 hours, 2 days, 3 days, 5 days, or 7 days after the test compound was administered to the subject.

### VII. Methods for Determining the State of a Disease Featuring Immune System Activation

Another aspect of the invention provides a method for determining whether certain medical disorders are in an active disease state or a dormant disease state. The method comprises evaluating the level of immune system activation in a subject, wherein minor differences in the abundance (e.g., less than approximately 5% wt/wt) and type of metabolites of ¹³C-labeled substrate (e.g., ¹³C-labeled glutamine, ¹³C-labeled palmitate, and ¹³C-labeled glucose) between a healthy subject and a diseased subject indicates that the disease is dormant, and larger differences in the abundance (e.g., greater than approximately 5% wt/wt) and type of metabolites of the ¹³C-labeled substrate between a healthy subject and a diseased subject indicates that the disease is active.

The level of immune system activation can be determined by evaluating the abundance and type of metabolite of any one or more of ¹³C-labeled glutamine, ¹³C-labeled palmitate, and ¹³C-labeled glucose according to the procedures described in any one of Sections I, II, or III above.

### VIII. Additional Methods

Another aspect of the invention provides a method for determining whether a therapeutic intervention to treat an immune disorder is working effectively. The method comprises administering medical therapy to a subject, and then testing for the presence of immune system activation in the subject according to any one of the methods described in Sections I and II above, wherein the absence of immune system activation in the subject is indicative that the medical therapy is working effectively. The method is contemplated to be applicable to the medical conditions listed in Section IV above, which are reiterated here. In certain embodiments, the step of testing for the presence of immune system activation is performed at least 1 hour, 5 hours, 12 hours, 2 days, 3 days, 5 days, or 7 days after administering medical therapy to the subject.

Another aspect of the invention provides a method for determining the amount of a therapeutic agent necessary to qualify as an effective amount to treat an immune disorder. The method comprises administering a therapeutic agent to a subject suffering from an immune disorder, and then testing for the presence of immune system activation in the subject according to any one of the methods described in Sections I and II above, wherein a reduction in immune system activation (or the absence of immune system activation) in the subject is indicative that the therapeutic agent was administered in an amount sufficient to qualify as an effective amount for treating said immune disorder. The method is contemplated to be applicable to the medical conditions listed in Section IV above, which are reiterated here. In certain embodiments, the step of testing for the presence of immune system activation is performed at least 1 hour, 5 hours, 12 hours, 2 days, 3 days, 5 days, or 7 days after administering the therapeutic agent to the subject.

Another aspect of the invention provides a method for determining an effective dosing regimen for treating an immune disorder using a therapeutic agent. The method comprises administering, according to a particular dosing regimen, a therapeutic agent to a subject suffering from an immune disorder, and then testing for the presence of immune system activation in the subject according to any one of the methods described in Sections I and II above, wherein a reduction in immune system activation (or the absence of immune system activation) in the subject is indicative that the therapeutic agent was administered using an effective dosing regimen. The method is contemplated to be applicable to the medical conditions listed in Section IV above, which are reiterated here. In certain embodiments, the step of testing for the presence of immune system activation is performed at least 1 hour, 5 hours, 12 hours, 2 days, 3 days, 5 days, or 7 days after administering the therapeutic agent to the subject.

### IX. Diagnostic Kits

Another aspect of the invention provides a kit for detecting the presence of immune system activation and/or a medical condition featuring immune system activation. The kit comprises (a) any one or more of ¹³C-labeled glutamine, ¹³C-labeled palmitate, and ¹³C-labeled glucose; and (b) instructions for determining the presence of immune system activation and/or a medical condition featuring immune system activation by comparing the abundance and/or type of metabolite of any one or more of ¹³C-labeled glutamine, ¹³C-labeled palmitate, and ¹³C-labeled glucose to a control value, such as control value specified in the instructions.

### X. Pharmaceutical Compositions Comprising ¹³C-Labeled Compounds

The ¹³C-labeled compounds may be formulated as a pharmaceutical composition for administration to the subject. Exemplary pharmaceutical compositions comprise one or more ¹³C-labeled compounds and one or more pharmaceutically acceptable carriers. As described in detail below, the pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

### EXAMPLES

The invention now being generally described, will be more readily understood by reference to the following examples, which are included merely for purposes of illustration of certain aspects and embodiments of the present invention, and are not intended to limit the invention.

### Example 1

The metabolite profiles of ¹³C-labeled glutamine, ¹³C-labeled potassium palmitate, and ¹³C-labeled glucose were evaluated in a mouse model of acute graft-versus-host disease. Experimental procedures are described below in Part A. The results are described below in Part B.

### Part A: Experimental Procedures

To investigate metabolic adaptations that occur in response to allo-activation, host strain animals (female B6D2F1 mice, H-2b/d) were injected with 50-75 million spleen cells from donor strain animals (female B6.SJL mice, H-2b). The miss-match between the strains major histocompatibility antigens (e.g., H-2b/d versus H-2b) causes T cell activation of donor T cells in vivo and results in a mouse model of acute graft versus host disease. Eight days after injection, transplanted mice along with control non-transplanted animals from the host and donor strains received with U-¹³C(6)-glutamine (Sigma#60516, 1.0 g/kg I.P.) or U-¹³C(5)-glucose (Sigma#660663, 1.0 g/kg I.P.) or potassium U-¹³C(16)-palmitate (Sigma#687871, 1.0 g/kg, gastric gavage). Treatment groups of four mice each were euthanized 60 minutes after the glutamine or glucose tracer loads or 180 minutes after palmitate tracer load by over exposure to carbon dioxide. Terminal blood samples were collected by cardiac puncture, plasma prepared after blood was mixed with heparin and frozen at −80° C. Spleens were removed and processed to isolate T cells as separate fractions derived from either the donor or host strains of mice. Samples were stored on dry ice overnight and then stored at −80° C. until analyzed using GC/MS-based mass isotopomer analysis.

### Part B: Result

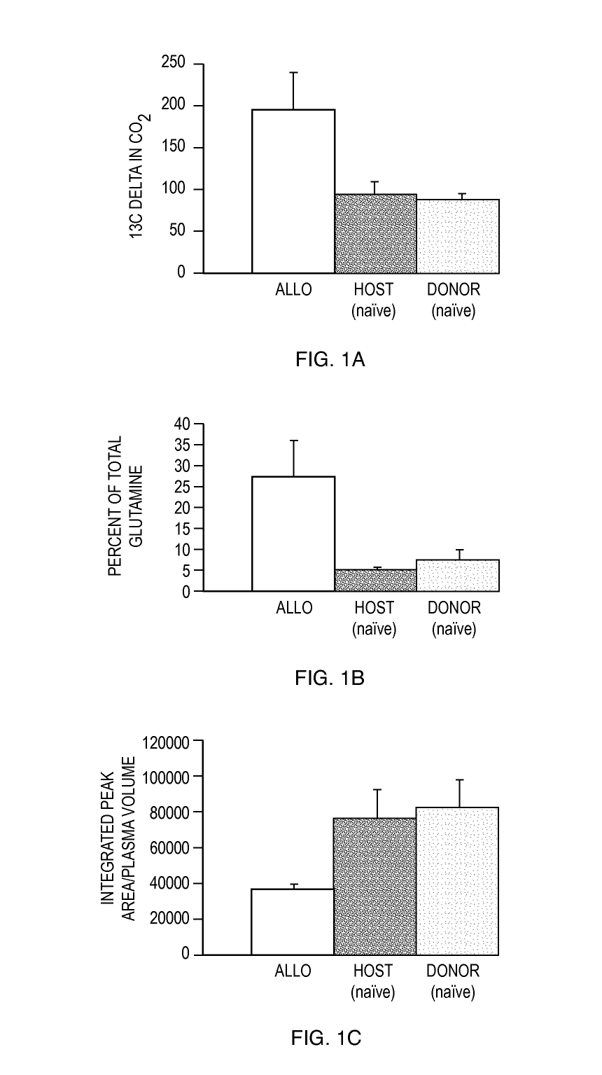

Results in FIG. 1 show that metabolic adaptations due to transplantation of allo-antigen miss-matched tissue resulted in the following features:

(A) an increased amount of complete oxidation of uniformly labeled ¹³C-glutamine to CO₂, as evidenced by the increase in ¹³C delta of CO₂ following ¹³C-glutamine tracer administration in allo-transplanted mice compared to control healthy donor mice and as compared to control healthy host mice;

(B) the labeled glutamine tracer (i.e., uniformly labeled ¹³C-glutamine) constituted a larger percentage of the total glutamine in plasma of allo-transplanted mice compared to control healthy donor mice and as compared to control healthy host mice; and

(C) a reduction in the concentration of total glutamine in plasma of allo-transplanted mice compared to control healthy donor mice and as compared to control healthy host mice.

The features identified above can be used as a plasma-based marker of allo-antigen miss-match induced alloimmune disease. Although not wishing to be bound by a particular theory, it is believed that these features indicate an increase in the amount of cycling type citric acid cycle metabolism in the subject. For example, on day 8, after injecting 50-75 million spleen cells from donor B6.SJL (H-2b, CD45.1⁺) mice into the tail vein of B6D2F1/J (H-2b/d, CD45.1⁻) hosts, we observed 197.81 ¹³CO₂ Δ (SD=37.92) in the plasma of grafted host animals in comparison with 89.89 ¹³CO₂ Δ (SD=24.75; p<0.01) in control donor and 87.52 ¹³CO₂ Δ (SD=7.85; p<0.01) of control host animals, respectively, using [U-¹³C₅]-L-glutamine (1 mg/g; I.P.) as the single tracer. Increased complete oxidation of glutamine in grafted host animals was accompanied by a more than two-fold drop in circulating total glutamine concentration, while glutamine's positional ¹³C analysis revealed a five-fold increase in its 13C-labeled fraction, of which 83.4% was still uniformly ¹³C-labeled and mostly undiluted, one hour after glutamine tracer load. These observations demonstrate that the effects of transplantation of allo-antigen miss-matched spleen cells that cause disease (e.g., acute and chronic graft versus host disease) can be measured by ¹³CO₂ output, glutamine consumption, and its limited recycling by the cycling citric acid cycle in host tissues with limited branching (which are the cycle's characteristics in control host and donor tissues). In addition, the close to five-fold surplus in circulating ¹³C-labeled injected glutamine in a single hour time frame provides a diagnostic and therapeutic efficacy tool for diseases featuring T-cell activation.

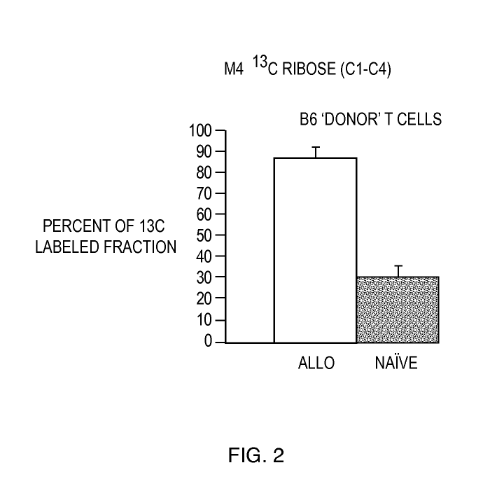

Results in FIG. 2 show that allo-antigen miss-matched transplantation causes donor T cells isolated from allo-antigen miss-matched mice to increase glutamine metabolism for ribose synthesis. For example, after administering uniformly labeled ¹³C-glutamine, the M4 fraction of ¹³C-labeled ribose increased in allo-activated T cells compared to control naïve (non-allo-activated) T cells. Thus, we have found that allo-transplantation causes a T cell to utilize glutamine for ribose synthesis. In addition, we have found that the quantity of M4 fraction of ¹³C-labeled ribose can used to evaluate disease activity.

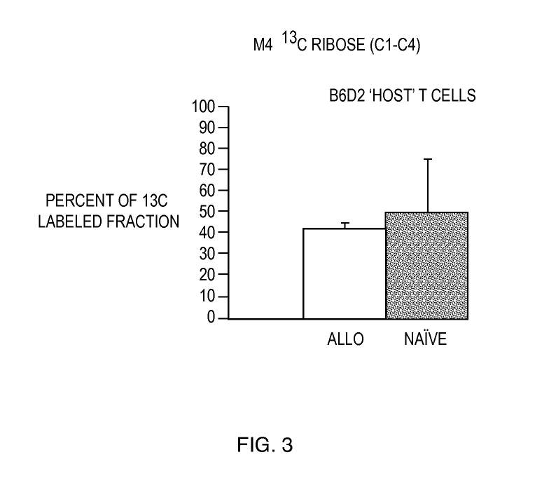

FIG. 3 shows that the change in glutamine metabolism occurs specifically in allo-activated T cells since there is no statistically significant difference in the percentage of M4 fraction of ribose between (i) host T cells from an allo-transplanted mouse (i.e., the host T cells are not allo-activated and do not cause disease) and (ii) T cells taken from a non-transplanted healthy host control animal (i.e., naïve subject). These observations demonstrate that transplantation of allo-antigen miss-matched spleen cells that cause disease (e.g., acute and chronic graft-versus-host disease) affects the conversion of glutamine into ribose in T cells. In particular, the three to five-fold increase in the percentage of ¹³C-labeled ribose in a single hour time frame provides a diagnostic and therapeutic efficacy tool for evaluating diseases featuring T-cell activation.

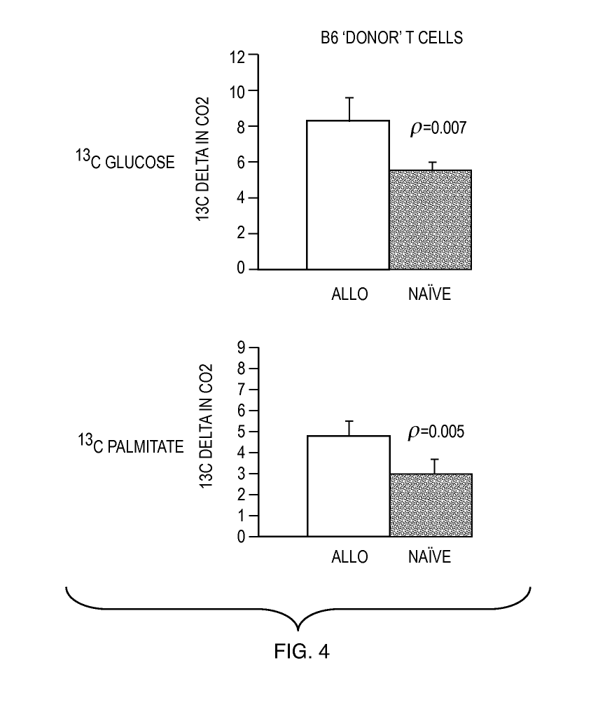

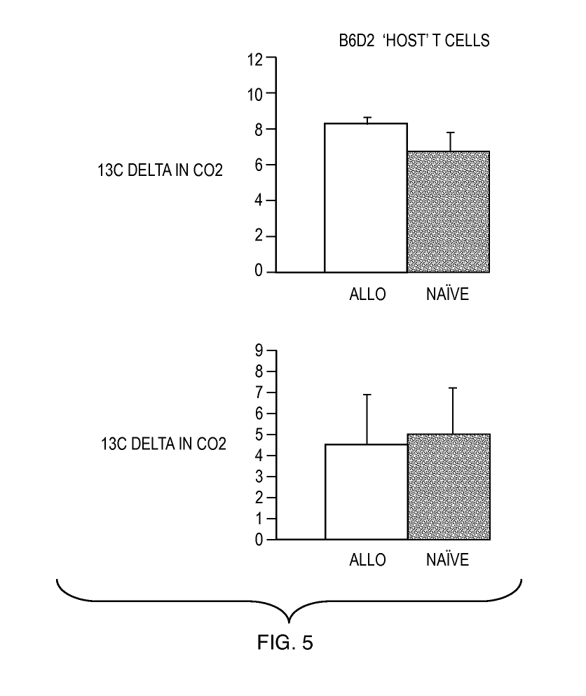

Results in FIG. 4 show that allo-antigen miss-matched spleen cell transplantation causes donor-derived allo-activated T cells isolated from allo-antigen miss-matched mice to increase oxidative metabolism of glucose and palmitate through pathways that result in complete oxidation to CO₂. For example, after administering either uniformly labeled ¹³C glucose or uniformly labeled ¹³C-palmitate, the ¹³C delta in CO₂ in allo-activated donor type T cells was higher than control naïve donor type T cells. This increase in the ¹³C delta in CO₂ in allo-activated donor type T cells is understood to be a T cell-based marker of allo-antigen activation. The increase in the ¹³C delta in CO₂ in allo-activated donor type T cells is also understood to identify enzymes and biochemical functions that constitute pathways of glucose and palmitate oxidation to CO₂ in T cells. Such enzymes and biochemical functions may be a target for new therapeutic agents useful for treating immune mediated diseases. The increase in the ¹³C delta in CO₂ in a single hour and three-hour time frame provides a diagnostic and therapeutic efficacy tool for evaluating diseases featuring T-cell activation. FIG. 5 shows that this change in oxidative metabolism occurs specifically in allo-activated T cells, since there is no statistically significant difference in the ¹³C delta in CO₂ between (i) host T cells from an allo-transplanted mouse (i.e., the host T cells are not allo-activated and do not cause disease) and (ii) host-type T cells taken from a non-transplanted healthy host control animal (i.e., naïve subject).

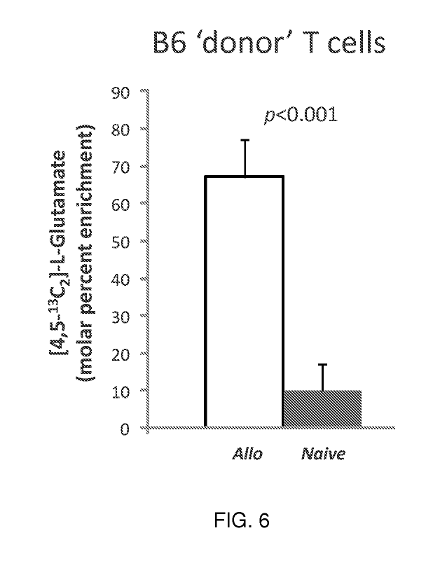

Results in FIG. 6 show that allo-antigen miss-matched transplantation causes donor T cells isolated from allo-antigen miss-matched mice to have a substantially higher molar percent enrichment of [4,5-¹³C₂]-L-glutamate compared to T cells taken from a non-transplanted healthy host control animal (i.e., naïve subject). For example, after administering uniformly labeled ¹³C-palmitate, the molar percent enrichment of [4,5-¹³C₂]-L-glutamate in allo-activated T cells was over six times greater than the molar percent enrichment of [4,5-¹³C₂]-L-glutamate observed in control naïve (non-allo-activated) T cells. The molar percent enrichment of [4,5-¹³C₂]-L-glutamate is the amount of [4,5-¹³C₂]-L-glutamate in a sample divided by the total amount of all ¹³C-labeled glutamate isotopomers in the sample.

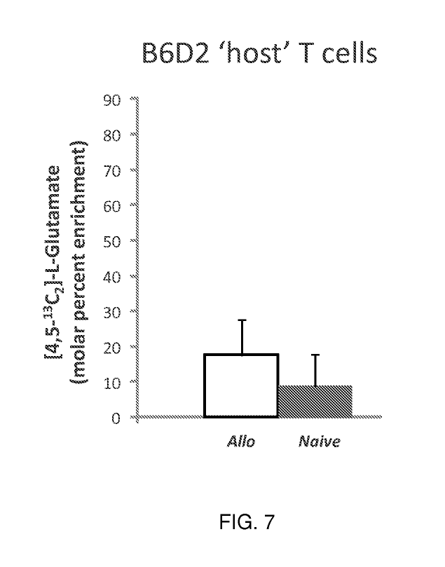

FIG. 7 shows that the increase in molar percent enrichment of [4,5-¹³C₂]-L-glutamate following administration of uniformly labeled ¹³C-palmitate occurs specifically in allo-activated T cells since there is no statistically significant difference in the molar percent enrichment of [4,5-¹³C₂]-L-glutamate between (i) host T cells from an allo-transplanted mouse (i.e., the host T cells are not allo-activated and do not cause disease) and (ii) T cells taken from a non-transplanted healthy host control animal (i.e., naïve subject).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of determining immune system activation in a subject, the method comprising one or more of the following steps:
(a) measuring the amount of ¹³CO₂ in a test sample obtained from a subject that received ¹³C-labeled glutamine; and comparing (i) the amount of ¹³CO₂ in the test sample to (ii) a control, wherein a greater amount of ¹³CO₂ in the test sample than the control indicates immune system activation in the subject;
(b) measuring the amount of ¹³C-labeled ribose in a population of cells selected from the group consisting of lymphocytes and immune cells, wherein said population of cells is obtained from a subject that received ¹³C-labeled glutamine; and comparing (i) the amount of ¹³C-labeled ribose in said population of cells to (ii) a control, wherein a greater amount of ¹³C-labeled ribose in said population of cells than the control indicates immune system activation in the subject;
(c) measuring the amount of ¹³C-labeled glutamine in a test sample obtained from a subject that received ¹³C-labeled glutamine; and comparing (i) the amount of ¹³C-labeled glutamine in the test sample to (ii) a control, wherein a greater amount of ¹³C-labeled glutamine in the test sample than the control indicates immune system activation in the subject; and
(d) measuring the amount of total glutamine in a test sample obtained from a subject that received glutamine; and comparing (i) the amount of total glutamine in the test sample to (ii) a control, wherein less total glutamine in the test sample than the control indicates immune system activation in the subject.

2. The method of claim 1, wherein the test sample is blood plasma.

3. The method of claim 1 or 2, wherein the ¹³C-labeled glutamine is glutamine where each carbon position contains ¹³C in an abundance of at least 75% wt/wt.

4. The method of claim 1 or 2, wherein the ¹³C-labeled glutamine is glutamine where each carbon position contains ¹³C in an abundance of at least 98% wt/wt.

5. The method of any one of claims 1-4, wherein the ¹³C-labeled glutamine is in the form of a free base.

6. The method of any one of claims 1-5, wherein the test sample was obtained from the subject about 30 minutes to about 90 minutes after ¹³C-labeled glutamine was administered to the subject.

7. The method of any one of claims 1-6, wherein the population of cells is a population of T-cells.

8. The method of claim 7, wherein the population of T-cells is obtained from blood, bone marrow, cells from bronchioalveolar lavage, or cells from a biopsy of a lymph node, a joint space, skin, or intestine of the subject.

9. The method of any one of claims 1-8, wherein the control is the average amount of corresponding analyte in samples taken from a population of healthy subjects subjected to approximately the same conditions as the subject from which the test sample was taken.

10. The method of any one of claims 1-9, wherein said comparing comprises performing statistical analysis using the Student's T-test.

11. The method of any one of claims 1-10, wherein measuring the amount of ¹³CO₂ in a test sample comprises analysis of the test sample using mass spectrometry to identify ¹³C-labeled carbon dioxide.

12. The method of any one of claims 1-11, wherein the method comprises step (a).

13. The method of any one of claims 1-12, wherein the method comprises step (b).

14. The method of any one of claims 1-13, wherein the method comprises step (c).

15. The method of any one of claims 1-14, wherein the method comprises step (d).

16. The method of any one of claims 1-15, wherein the immune system activation comprises T-cell activation.

17. The method of any one of claims 1-15, wherein the immune system activation comprises activation of one or more of T-cells, B-cells, monocytes, macrophages, peripheral blood mononuclear cells, or peripheral blood mononuclear leukocytes.

18. A method of determining immune system activation in a subject, the method comprising one or more of the following steps:
(a) measuring the amount of ¹³CO₂ in a test sample obtained from a subject that received ¹³C-labeled palmitate; and comparing (i) the amount of ¹³CO₂ in the test sample to (ii) a control, wherein a greater amount of ¹³CO₂ in the test sample than the control indicates immune system activation in the subject; and
(b) measuring the molar percent enrichment in [4,5-¹³C₂]-L-glutamate in a test sample obtained from a subject that received ¹³C-labeled palmitate; and comparing (i) the molar percent enrichment in [4,5-¹³C₂]-L-glutamate in the test sample to (ii) a control, wherein greater molar percent enrichment in [4,5-¹³C₂]-L-glutamate in the test sample compared to the control indicates immune system activation in the subject.

19. The method claim 18, wherein the method comprises step (a).

20. The method of claim 18 or 19, wherein the method comprises step (b).

21. The method of any one of claims 18-20, wherein the test sample is a population of T-cells obtained from a subject that received ¹³C-labeled palmitate.

22. The method of claim 21, wherein the population of T-cells is obtained from blood, bone marrow, cells from bronchioalveolar lavage, or cells from a biopsy of a lymph node, a joint space, skin, or intestine of the subject.

23. The method of any one of claims 18-20, wherein the test sample is a population of lymphocytes or immune cells, each of which are obtained from a subject that received ¹³C-labeled palmitate.

24. The method of any one of claims 18-23, wherein the ¹³C-labeled palmitate is palmitate where each carbon position contains ¹³C in an abundance of at least 75% wt/wt.

25. The method of any one of claims 18-23, wherein the ¹³C-labeled palmitate is palmitate where each carbon position contains ¹³C in an abundance of at least 98% wt/wt.

26. The method of any one of claims 18-25, wherein the ¹³C-labeled palmitate is an alkali metal ¹³C-labeled palmitate salt.

27. The method of any one of claims 18-25, wherein the ¹³C-labeled palmitate is potassium ¹³C-labeled palmitate.

28. The method of any one of claims 18-27, wherein the test sample was obtained from the subject about 2 hours to about 4 hours after ¹³C-labeled palmitate was administered to the subject.

29. The method of any one of claims 18-28, wherein the control in step (a) is the average amount of ¹³CO₂ in samples taken from a population of healthy subjects subjected to approximately the same conditions as the subject from which the test sample was taken.

30. The method of any one of claims 18-29, wherein said comparing comprises performing statistical analysis using the Student's T-test.

31. The method of any one of claims 18-30, wherein measuring the amount of ¹³CO₂ in a test sample comprises analysis of the test sample using mass spectrometry to identify ¹³C-labeled carbon dioxide.

32. The method of any one of claims 18-31, further comprising the steps of:
(a-1) measuring the amount of ¹³CO₂ in a test sample obtained from a subject that received ¹³C-labeled glucose; and
(b-1) comparing (i) the amount of ¹³CO₂ in the test sample to (ii) a control, wherein a greater amount of ¹³CO₂ in the test sample than the control indicates immune system activation in the subject.

33. The method of claim 32, wherein the test sample in steps a-1 and b-1 is a population of T-cells obtained from said subject that received ¹³C-labeled glucose.

34. The method of claim 32 or 33 wherein the ¹³C-labeled glucose is glucose where each carbon position contains ¹³C in an abundance of at least 75% wt/wt.

35. The method of claim 32 or 33, wherein the ¹³C-labeled glucose is glucose where each carbon position contains ¹³C in an abundance of at least 98% wt/wt.

36. The method of any one of claims 32-35, wherein the test sample was obtained from the subject about 30 minutes to about 90 minutes after ¹³C-labeled glucose was administered to the subject.

37. The method of any one of claims 32-36, wherein the control in step b-1 is the average amount of ¹³CO₂ in samples taken from a population of healthy subjects subjected to approximately the same conditions as the subject from which the test sample was taken.

38. A method of determining whether a subject has a medical condition featuring immune system activation, the method comprising one or more of the following steps:
(a) measuring the amount of ¹³CO₂ in a test sample obtained from a subject that received ¹³C-labeled glutamine; and comparing (i) the amount of ¹³CO₂ in the test sample to (ii) a control, wherein a greater amount of ¹³CO₂ in the test sample than the control indicates that the subject has the medical condition;
(b) measuring the amount of ¹³C-labeled ribose in a population of cells selected from the group consisting of lymphocytes and immune cells, wherein said population of cells is obtained from a subject that received ¹³C-labeled glutamine; and comparing (i) the amount of ¹³C-labeled ribose in said population of cells to (ii) a control, wherein a greater amount of ¹³C-labeled ribose in said population of cells than the control indicates that the subject has the medical condition;
(c) measuring the amount of ¹³C-labeled glutamine in a test sample obtained from a subject that received ¹³C-labeled glutamine; and comparing (i) the amount of ¹³C-labeled glutamine in the test sample to (ii) a control, wherein a greater amount of ¹³C-labeled glutamine in the test sample than the control indicates that the subject has the medical condition; and
(d) measuring the amount of total glutamine in a test sample obtained from a subject that received glutamine; and comparing (i) the amount of total glutamine in the test sample to (ii) a control, wherein less total glutamine in the test sample than the control indicates that the subject has the medical condition.

39. The method of claim 38, wherein the test sample is blood plasma.

40. The method of claim 38 or 39, wherein the ¹³C-labeled glutamine is glutamine where each carbon position contains ¹³C in an abundance of at least 75% wt/wt.

41. The method of claim 38 or 39, wherein the ¹³C-labeled glutamine is glutamine where each carbon position contains ¹³C in an abundance of at least 98% wt/wt.

42. The method of any one of claims 38-41, wherein the ¹³C-labeled glutamine is in the form of a free base.

43. The method of any one of claims 38-42, wherein the test sample was obtained from the subject about 30 minutes to about 90 minutes after ¹³C-labeled glutamine was administered to the subject.

44. The method of any one of claims 38-43, wherein the population of cells is a population of T-cells.

45. The method of claim 44, wherein the population of T-cells is obtained from blood, bone marrow, cells from bronchioalveolar lavage, or cells from a biopsy of a lymph node, a joint space, skin, or intestine of the subject.

46. The method of any one of claims 38-45, wherein the control is the average amount of corresponding analyte in samples taken from a population of healthy subjects subjected to approximately the same conditions as the subject from which the test sample was taken.

47. The method of any one of claims 38-46, wherein said comparing comprises performing statistical analysis using the Student's T-test.

48. The method of any one of claims 38-47, wherein measuring the amount of ¹³CO₂ in a test sample comprises analysis of the test sample using mass spectrometry to identify ¹³C-labeled carbon dioxide.

49. The method of any one of claims 38-48, wherein the method comprises step (a).

50. The method of any one of claims 38-49, wherein the method comprises step (b).

51. The method of any one of claims 38-50, wherein the method comprises step (c).

52. The method of any one of claims 38-51, wherein the method comprises step (d).

53. A method of determining whether a subject has a medical condition featuring immune system activation, the method comprising one or more of the following steps:
(a) measuring the amount of ¹³CO₂ in a test sample obtained from a subject that received ¹³C-labeled palmitate; and comparing (i) the amount of ¹³CO₂ in the test sample to (ii) a control, wherein a greater amount of ¹³CO₂ in the test sample than the control indicates that the subject has the medical condition; and
(b) measuring the molar percent enrichment in [4,5-¹³C₂]-L-glutamate in a test sample obtained from a subject that received ¹³C-labeled palmitate; and comparing (i) the molar percent enrichment in [4,5-¹³C₂]-L-glutamate in the test sample to (ii) a control, wherein greater molar percent enrichment in [4,5-¹³C₂]-L-glutamate in the test sample compared to the control indicates that the subject has the medical condition.

54. The method claim 53, wherein the method comprises step (a).

55. The method of claim 53 or 54, wherein the method comprises step (b).

56. The method of any one of claims 53-55, wherein the ¹³C-labeled palmitate is palmitate where each carbon position contains ¹³C in an abundance of at least 75% wt/wt.

57. The method of any one of claims 53-55, wherein the ¹³C-labeled palmitate is palmitate where each carbon position contains ¹³C in an abundance of at least 98% wt/wt.

58. The method of any one of claims 53-57, wherein the ¹³C-labeled palmitate is an alkali metal ¹³C-labeled palmitate salt.

59. The method of any one of claims 53-57, wherein the ¹³C-labeled palmitate is potassium ¹³C-labeled palmitate.

60. The method of any one of claims 53-59, wherein the test sample was obtained from the subject about 2 hours to about 4 hours after ¹³C-labeled palmitate was administered to the subject.

61. The method of any one of claims 53-60, wherein the test sample is a population of T-cells obtained from a subject that received ¹³C-labeled palmitate.

62. The method of claim 61, wherein the population of T-cells is obtained from blood, bone marrow, cells from bronchioalveolar lavage, or cells from a biopsy of a lymph node, a joint space, skin, or intestine of the subject.

63. The method of any one of claims 53-60, wherein the test sample is a population of lymphocytes or immune cells, each of which is obtained from a subject that received ¹³C-labeled palmitate.

64. The method of any one of claims 53-63, wherein the control in step (a) is the average amount of ¹³CO₂ in samples taken from a population of healthy subjects subjected to approximately the same conditions as the subject from which the test sample was taken.

65. The method of any one of claims 53-64, wherein said comparing comprises performing statistical analysis using the Student's T-test.

66. The method of any one of claims 53-65, wherein measuring the amount of ¹³CO₂ in a test sample comprises analysis of the test sample using mass spectrometry to identify ¹³C-labeled carbon dioxide.

67. The method of any one of claims 38-66, wherein the medical condition featuring immune system activation is an immune disorder.

68. The method of claim 67, wherein the immune disorder is chronic graft-versus-host disease, acute graft-versus-host disease, rheumatoid arthritis, psoriasis, Crohn's disease, inflammatory bowel disease, multiple sclerosis, systemic lupus erythematosus, Celiac Sprue, idiopathic thrombocytopenic thrombotic purpura, myasthenia gravis, Sjogren's syndrome, scleroderma, ulcerative colitis, asthma, uveitis, or epidermal hyperplasia.

69. The method of any one of claims 38-66, wherein the medical condition featuring immune system activation is cartilage inflammation, bone degradation, arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, dermatomyositis, psoriatic arthritis, vasculitis, myolitis, polymyolitis, dermatomyolitis, osteoarthritis, polyarteritis nodossa, Wegener's granulomatosis, arteritis, polymyalgia rheumatica, sarcoidosis, sclerosis, primary biliary sclerosis, sclerosing cholangitis, dermatitis, atopic dermatitis, atherosclerosis, Still's disease, chronic obstructive pulmonary disease, Guillain-Barre disease, Type I diabetes mellitus, Graves' disease, Addison's disease, Raynaud's phenomenon, lupus nephritis, glomerular nephritis, or autoimmune hepatitis.

70. The method of any one of claims 38-66, wherein the medical condition featuring immune system activation is muscular dystrophy, arthritis, traumatic brain injury, spinal cord injury, sepsis, rheumatic disease, cancer atherosclerosis, type 1 diabetes, type 2 diabetes, leptospiriosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, ischemia/reperfusion, stroke, cerebral aneurysm, angina pectoris, pulmonary disease, cystic fibrosis, acid-induced lung injury, pulmonary hypertension, asthma, chronic obstructive pulmonary disease, Sjogren's syndrome, hyaline membrane disease, kidney disease, glomerular disease, alcoholic liver disease, gut diseases, peritoneal endometriosis, skin diseases, nasal sinusitis, mesothelioma, anhidrotic ecodermal dysplasia-ID, behcet's disease, incontinentia pigmenti, tuberculosis, asthma, crohn's disease, colitis, ocular allergy, appendicitis, paget's disease, pancreatitis, periodonitis, endometriosis, inflammatory bowel disease, inflammatory lung disease, silica-induced diseases, sleep apnea, AIDS, HIV-1, autoimmune diseases, antiphospholipid syndrome, lupus, lupus nephritis, familial mediterranean fever, hereditary periodic fever syndrome, psychosocial stress diseases, neuropathological diseases, familial amyloidotic polyneuropathy, inflammatory neuropathy, parkinson's disease, multiple sclerosis, alzheimer's disease, amyotropic lateral sclerosis, huntington's disease, cataracts, and hearing loss.

71. The method of any one of claims 38-66, wherein the medical condition featuring immune system activation is head injury, uveitis, inflammatory pain, allergen induced asthma, non-allergen induced asthma, glomerular nephritis, ulcerative colitis, necrotizing enterocolitis, hyperimmunoglobulinemia D with recurrent fever (HIDS), TNF receptor associated periodic syndrome (TRAPS), cryopyrin-associated periodic syndromes, Muckle-Wells syndrome (urticaria deafness amyloidosis), familial cold urticaria, neonatal onset multisystem inflammatory disease (NOMID), periodic fever, aphthous stomatitis, pharyngitis and adenitis (PFAPA syndrome), Blau syndrome, pyogenic sterile arthritis, pyoderma gangrenosum, acne (PAPA), deficiency of the interleukin-1-receptor antagonist (DIRA), subarachnoid hemorrhage, polycystic kidney disease, transplant, organ transplant, tissue transplant, myelodysplastic syndrome, irritant-induced inflammation, plant irritant-induced inflammation, poison ivy/urushiol oil-induced inflammation, chemical irritant-induced inflammation, bee sting-induced inflammation, insect bite-induced inflammation, sunburn, burns, dermatitis, endotoxemia, lung injury, acute respiratory distress syndrome, alcoholic hepatitis, and kidney injury caused by a parasitic infection.

72. The method of any one of claims 38-71, further comprising the steps of:
(a-1) measuring the amount of ¹³CO₂ in a test sample obtained from a subject that received ¹³C-labeled glucose; and
(b-1) comparing (i) the amount of ¹³CO₂ in the test sample to (ii) a control, wherein a greater amount of ¹³CO₂ in the test sample than the control indicates that the subject has said immune disorder.

73. A method of determining whether a subject has an immune disorder selected from the group consisting of graft-versus-host disease, acute graft-versus-host disease, rheumatoid arthritis, psoriasis, Crohn's disease, inflammatory bowel disease, multiple sclerosis, Celiac Sprue, idiopathic thrombocytopenic thrombotic purpura, myasthenia gravis, Sjogren's syndrome, scleroderma, ulcerative colitis, asthma, uveitis, epidermal hyperplasia, cartilage inflammation, bone degradation, arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome, juvenile dermatomyositis, juvenile psoriatic arthritis, juvenile scleroderma, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, dermatomyositis, psoriatic arthritis, vasculitis, myolitis, polymyolitis, dermatomyolitis, osteoarthritis, polyarteritis nodossa, Wegener's granulomatosis, arteritis, polymyalgia rheumatica, sarcoidosis, sclerosis, primary biliary sclerosis, sclerosing cholangitis, dermatitis, atopic dermatitis, atherosclerosis, Still's disease, chronic obstructive pulmonary disease, Guillain-Barre disease, Type I diabetes mellitus, Graves' disease, Addison's disease, Raynaud's phenomenon, glomerular nephritis, or autoimmune hepatitis, the method comprising the steps of:
(a) measuring the amount of ¹³CO₂ in a test sample obtained from a subject that received ¹³C-labeled glucose; and
(b) comparing (i) the amount of ¹³CO₂ in the test sample to (ii) a control, wherein a greater amount of ¹³CO₂ in the test sample than the control indicates that the subject has said immune disorder.
